(19) United States
(12) Patent Application Publication
(10) Pub. No.: US 2026/0272278 A1
(43) Pub. Date: Sep. 17, 2026

(54) METHOD AND SYSTEM FOR ORAL IMAGING

(71) Applicant: Enamel Pure Inc., Worcester, MA (US)

(72) Inventors: Charles Holland Dresser, Wayland, MA (US); Nathan Paul Monty, Shrewsbury, MA (US)

(21) Appl. No.: 19/080,986

(22) Filed: Mar. 17, 2025

Related U.S. Application Data
(60) Provisional application No. 63/565,119, filed on Mar. 14, 2024.

(51) Int. Cl. A61B 1/247; A61B 1/00; A61C 17/18

(52) U.S. Cl. CPC A61B 1/247; A61B 1/00188; A61C 17/18

(57) ABSTRACT

Disclosed are a system and a method for oral imaging. The system includes a hand piece, an optical arrangement, and a sensor. The hand piece includes a distal optical passage and a proximal optical passage. The distal optical passage is located at a distal end of the hand piece configured to be located within a patient's mouth. The proximal optical passage is located at the proximal end of the hand piece configured to be located outside of the patient's mouth. The optical arrangement is configured in optical communication with the proximal optical passage and the distal optical passage. The optical arrangement is configured to view dental tissue, via the proximal optical passage and the distal optical passage. The lens arrangement is configured to form an optical image of the dental tissue. The sensor is connected in optical communication with the optical arrangement and configured to detect the optical image of the dental tissue. The sensor is further configured to generate a digital image of the dental tissue, as a function of the optical image of the dental tissue.

### FIELD OF THE INVENTION

The present invention generally relates to the field of oral imaging. In particular, the present invention is directed to a method and a system for oral imaging.

### BACKGROUND

There is no way to simultaneously image teeth concurrently with dental cleanings.

### SUMMARY OF THE DISCLOSURE

Laser tissue treatment necessitates an optical path to reach the treatment site. Throughout the treatment, such as laser cleaning, this optical path covers every treated tissue region. Consequently, an imaging system in optical communication with the laser's optical path is directed at each treated tissue region. In a complete dental laser cleaning or purification procedure, the imaging system captures images of every dental surface. However, geometric constraints within a dental laser device limit the imaging system's placement. Typically, intraoral cameras and scanners have optical assemblies and image sensors located within or very near the patient's mouth. In this application, the goal is to position the imaging system, including the optical assembly and image sensor, as far away from the patient as possible to minimize the instrument's size in the patient's mouth. Achieving this necessitates a relatively long working distance. While long working distances entail design trade-offs, telecentric imaging proves suitable. Telecentric imaging, commonly used in machine vision applications, provides constant magnification over the entire depth of field (DOF). This results in images without perspective, making them easier for computer analysis, particularly for high-resolution two-dimensional measurements.

The present disclosure recognizes that there is a need for a system and method capable of capturing clinically meaningful images for use in dental laser treatments. Specifically, it focuses on creating a solution that enhances 2D AI, 3D AI, and optical measurement for defining clear aligners. The challenge lies in optimizing the imaging system's placement, including the optical assembly and image sensor, within the constraints of a dental laser device. By emphasizing a long working distance and leveraging telecentric imaging, the present disclosure aims to provide clear and precise images of dental surfaces, contributing to improved treatment outcomes in dental laser procedures and enhancing the capabilities of AI-based and optical measurements for orthodontic purposes.

Thus, in view of the above, there is a long-felt need in the industry to address the aforementioned deficiencies and inadequacies.

The approaches described in this section are approaches that could be pursued, but these are not necessarily approaches that have been previously conceived or pursued. Therefore, unless otherwise indicated, it should not be assumed that any of the approaches described in this section qualify as prior art merely by virtue of their inclusion in this section.

In an aspect, a system for oral imaging is provided. The system includes a hand piece, an optical arrangement, and a sensor. The hand piece includes a distal optical passage and a proximal optical passage. The distal optical passage is located at a distal end of the hand piece configured to be located within a patient's mouth. The proximal optical passage is located at the proximal end of the hand piece configured to be located outside of the patient's mouth. The optical arrangement is configured in optical communication with the proximal optical passage and the distal optical passage. The optical arrangement is configured to view dental tissue, via the proximal optical passage and the distal optical passage. The lens arrangement is configured to form an optical image of the dental tissue. The sensor is connected in optical communication with the optical arrangement and configured to detect the optical image of the dental tissue. The sensor is further configured to generate a digital image of the dental tissue, as a function of the optical image of the dental tissue.

In additional system embodiments, the optical arrangement comprises a telecentric optical.

In additional system embodiments, the optical arrangement comprises an adaptive optic configured to adjust a working distance of the optical arrangement.

In additional system embodiments, the optical arrangement is located at least 50 mm, 100 mm, 150 mm, 200 mm, 250 mm, or 500 mm from the distal optical passage.

In additional system embodiments, the system includes a laser, and a beam delivery system. The laser is configured to generate a laser beam as a function of a laser parameter. The laser beam performs a treatment on the dental tissue, as a function of the laser parameter. The beam delivery system is in optical communication with the laser and the distal optical passage. The beam delivery system is configured to deliver the laser beam from the laser to the hand piece. The hand piece is configured to accept the laser beam from the beam delivery system at the distal optical passage. The hand piece is further configured to direct the laser beam, using the proximal optical passage, to the dental hard tissue.

In additional system embodiments, the beam delivery system includes at least a portion of the optical arrangement.

In additional system embodiments, the sensor has a shutter synchronized to pulses of the laser.

In additional system embodiments, the beam delivery system includes a beam scanning system configured to move the laser beam. The sensor has a shutter synchronized to at least a movement of the beam scanning system.

In additional system embodiments, the optical arrangement includes an adaptive optic configured to adjust a working distance of the optical arrangement. The sensor has shutter synchronized to the adaptive optic and the adjustment of the working distance of the optical arrangement.

In additional system embodiments, the sensor further includes a computing device configured to control one or more of the laser, the sensor, the shutter, the adaptive optic, an illumination source, and the beam scanning system.

In another aspect, a method for oral imaging is disclosed. The method includes a step of locating, using a hand piece, a distal optical passage located at a distal end of the hand piece within a patient's mouth. The method includes a step of locating, using the hand piece, a proximal optical passage located at a proximal end of the hand piece outside of the patient's mouth. The method includes a step of viewing at the dental tissue using an optical arrangement connected to both the proximal and distal optical passages. The method includes a step of forming, using the optical arrangement, an optical image of the dental tissue. The method includes a step of detecting, using a sensor in optical communication with the optical arrangement, the optical image of the dental tissue. The method includes a step of generating, using the sensor, a digital image of the dental tissue, as a function of the optical image of the dental tissue.

In additional method embodiments, the optical arrangement comprises a telecentric optical.

In additional method embodiments, the method includes a step of adjusting, using the optical arrangement comprising an adaptive optic, a working distance of the optical arrangement.

In additional method embodiments, the optical arrangement is located at least 50 mm, 100 mm, 150 mm, 200 mm, 250 mm, or 500 mm from the distal optical passage.

In additional method embodiments, the method includes a step of generating, using a laser, a laser beam as a function of a laser parameter, wherein the laser beam performs a treatment on the dental tissue, as a function of the laser parameter.

In additional method embodiments, the method includes a step of delivering the laser beam from the laser to the hand piece by using a beam delivery system.

In additional method embodiments, the method includes a step of accepting, using the hand piece, the laser beam from the beam delivery system at the distal optical passage.

In additional method embodiments, the method includes a step of directing the laser beam, using the proximal optical passage of the hand piece, to the dental hard tissue.

In additional method embodiments, the beam delivery system comprises at least a portion of the optical arrangement.

In additional method embodiments, the method includes a step of synchronizing a shutter of the sensor to pulses of the laser.

In additional method embodiments, the method includes a step of moving, using a beam scanning system of the beam delivery system, the laser beam.

In additional method embodiments, the method includes a step of synchronizing a shutter of the sensor to at least a movement of the beam scanning system.

In additional method embodiments, the method includes a step of adjusting a working distance of the optical arrangement, using an adaptive optic.

In additional method embodiments, the method includes a step of synchronizing a shutter of the sensor to the adaptive optic and the adjustment of the working distance of the optical arrangement.

In additional method embodiments, the sensor further includes a computing device configured to control one or more of the laser, the sensor, the shutter, the adaptive optic, an illumination source, and the beam scanning system.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 illustrates a perspective view of a system for oral imaging, in accordance with an embodiment of the present subject matter.

FIG. 2 illustrates an exemplary system, in accordance with an embodiment of the present subject matter.

FIG. 3A illustrates a view of a dental laser hand piece with an attachable tip, in accordance with an embodiment of the present subject matter.

FIG. 3B illustrates a cross-sectional view of a dental laser hand piece with an attachable tip of FIG. 3A, in accordance with an embodiment of the present subject matter.

FIG. 4A illustrates a hand piece with a consumable sheath attached, in accordance with an embodiment of the present subject matter.

FIG. 4B illustrates a cross-sectional view of the hand piece and the sheath of FIG. 4A.

FIG. 5 illustrates an experimental view of the tooth imaging, in accordance with an embodiment of the present subject matter.

FIG. 6 illustrates a flow diagram illustrating a method for oral imaging, in accordance with an embodiment of the present subject matter.

FIGS. 7A-7B illustrate graphical representations of transmission data of KCl and KBr, in accordance with an embodiment of the present subject matter.

FIG. 8 illustrates a layout and spot diagram of the separated KBr-CLEARTRAN achromat, in accordance with an embodiment of the present subject matter.

FIG. 9 illustrates a layout and spot diagram of the separated KBr-CLEARTRAN achromat on angular space, in accordance with an embodiment of the present subject matter.

FIG. 10 illustrates a layout of an original system with the redesigned lens, in accordance with an embodiment of the present subject matter.

FIG. 11 illustrates a graphical representation of poly MTF of the original system with the achromatized lens, in accordance with an embodiment of the present subject matter.

FIG. 12 illustrates a layout of the combined 8f relay system, in accordance with an embodiment of the present subject matter.

FIG. 13 illustrates a layout of case O₃, where the mirror for imaging purpose is placed after the 4th relay lens, in accordance with an embodiment of the present subject matter.

FIG. 14 illustrates a graphical representation of poly MTF of case O3, in accordance with an embodiment of the present subject matter.

FIG. 15 illustrates a layout of case O₂, where the relay lens is responsible for imaging, in accordance with an embodiment of the present subject matter.

FIG. 16 illustrates a poly MTF of case O₃, in accordance with an embodiment of the present subject matter.

FIG. 17 illustrates a layout of the combined 8f relay system with all relay lens replaced by doublets, in accordance with an embodiment of the present subject matter.

FIG. 18 illustrates a spot diagram of the CO₂ beam through the achromatic 8f system, in accordance with an embodiment of the present subject matter.

The drawings are not necessarily to scale and may be illustrated by phantom lines, diagrammatic representations, and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for oral imaging. The system includes a hand piece, an optical arrangement, and a sensor. The hand piece has a distal optical passage and a proximal optical passage, with the distal optical passage situated at the handpiece's end within a patient's mouth and the proximal passage located at the hand piece's outer end. The optical arrangement connects with both passages and is designed to observe dental tissue through them, forming an optical image. A sensor, linked to the optical arrangement, detects the optical image and generates a digital representation of the dental tissue based on the optical image.

Aspects of the present disclosure can be used to capture clinically meaningful images for use in dental laser treatments. Aspects of the present disclosure can also be used to create a solution that enhances 2D artificial intelligence (AI), 3D AI, and optical measurement for defining clear aligners.

Typically, a difficulty involves strategically positioning the imaging system components, such as the optical assembly and image sensor, a patient's mouth and/or within the limitations of existing dental laser device. This disclosure emphasizes achieving an extended working distance and integrating telecentric imaging. An objective may be to deliver sharp and accurate images of dental surfaces, thereby enhancing effectiveness of dental laser procedures and elevating the potential for AI-based applications and optical measurements, for instance without limitations in an orthodontic context. This optimization seeks to significantly contribute to advancing treatment outcomes and overall capabilities in dental imaging technologies. Exemplary embodiments illustrating aspects of the present disclosure are described below in the context of several specific examples.

An image may be optical, such as without limitation where at least an optic (e.g., optical arrangement) is used to generate an image of an object. An image may be material, such as without limitation when film is used to capture an image. An image may be digital, such as without limitation when represented as a bitmap. Alternatively, an image may be comprised of any media capable of representing a physical scene, space, and/or object. Alternatively, where “image” is used as a verb, in this disclosure, it refers to the generation and/or formation of an image.

As used in this disclosure, “dental tissue” refers to any tissue within a mouth, for instance, dental hard tissue (e.g., enamel, dentin, cementum, and osseous tissue) and dental soft tissue (e.g., gums, mucosa, and tongue).

As used in this disclosure, a “sensor” refers to any component, system, or device that detects a phenomenon, such as an optical image sensor that detects a radiative phenomenon like presence of light. As used in this disclosure, an “optical image sensor” is a device that is configured to sense electromagnetic radiation, such as without limitation visible light, and generate an image representing the electromagnetic radiation. In some cases, an optical image sensor may work in conjunction with one or more optics (e.g., optical arrangement). In some cases, at least an optical image may include an optical sensor. Exemplary non-limiting optical sensors include digital image sensors, such as without limitation charge-coupled device (CCD) sensors and complementary metal-oxide-semiconductor (CMOS) sensors, chemical image sensors, and analog image sensors, such as without limitation photodiodes and thermopiles. In some cases, an optical image sensor may be sensitive within a non-visible range of electromagnetic radiation, such as without limitation infrared. In some cases, an optical image sensor, or sensor generally, may have a shutter. As used in this disclosure, a “shutter” is a component or feature that temporally modulates function of a component, system, or device, for instance, an optical image sensor. A shutter may determine frame time for an optical image sensor. A shutter may be global, permitting detection by all sensing components on an optical image sensor simultaneously. Alternatively or additionally, an optical image sensor may be rolling permitting detection by only some (e.g., a row) of sensing components on an optical image sensor simultaneously.

As used in this disclosure, an “adaptive optic” is an optic with controllably variable optical properties, such as electro-optic lenses, liquid lenses, spatial light modulators, and the like. In some cases, an adaptive optic may be used to vary one or more of optical power, working distance, focal length, or the like of optical arrangement.

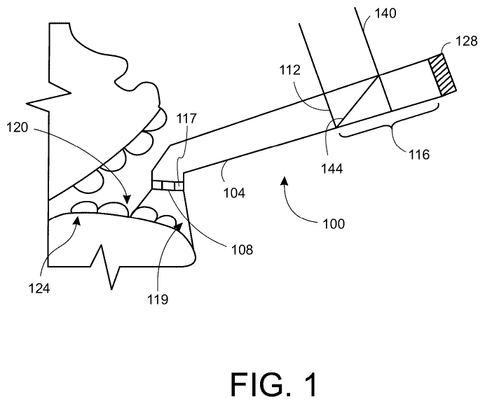

Referring now to FIG. 1, an exemplary embodiment of a perspective view of a system 100 for oral imaging is illustrated. System 100 may include a hand piece 104, an optical arrangement 116, and a sensor 128. Hand piece 104 includes a distal optical passage 108 and a proximal optical passage 112. As used in this disclosure, a “hand piece” is a component that is configured to be held in one's hand, for instance, held in the hand of a dental practitioner, such as a dental hygienist. As used in this disclosure, an “optical passage” is a component and/or feature of a component that allows for the passage of light. For example, an optical passage may include an aperture, a window, a lens, a prism, a waveguide, a reflector, or the like. In some cases, an optical passage may allow for light to pass through (e.g., into and/or out of) a component. For instance, in some cases, a hand piece may have a distal optical passage located at a distal end of the hand piece allowing for light passage into and/or out of a distal end of the hand piece. Alternatively or additionally, hand piece may have a proximal optical passage located at a proximal end of hand piece allowing for light passage into and/or out of a distal end of hand piece. As used in this disclosure, an “optical arrangement” is any component, assembly, system, or device that provides an optical function, such as an optical assembly or even a single optic. Exemplary non-limiting optics include spherical lenses, aspherical lenses, reflectors, polarizers, filters, windows, aperture stops, and the like. In some cases, an optical arrangement may configured to view an object and form a corresponding optical image. “Image,” where used as a noun in this disclosure, refers to a representation of at least a physical scene, space, and/or object (e.g., dental tissue). As used in this disclosure, an “optical image” is optical (e.g., radiation or light-based) information representing at least a physical scene, space, and/or object. As used in this disclosure, a “digital image” is digital information representing at least a physical scene, space, and/or object (e.g., dental tissue). In some cases, a digital image may be generated by a camera. Distal optical passage 108 is located at a distal end of the hand piece 104 configured to be located within a patient's mouth 124. Proximal optical passage 112 is located at proximal end of hand piece 104 configured to be located outside of the patient's mouth 124. Optical arrangement 116 is configured in optical communication with the proximal optical passage 112 and the distal optical passage 108. In some embodiments, optical arrangement 116 may provide optical communication between the proximal optical passage 112 and the distal optical passage 108. The optical arrangement is configured to view dental tissue 120, via the proximal optical passage 112 and the distal optical passage 108. Lens arrangement is configured to form an optical image of the dental tissue 120. The sensor 128 is connected in optical communication with the optical arrangement 116 and configured to detect the optical image of the dental tissue 120. In an embodiment, the optical arrangement 116 comprises a telecentric optical. In an embodiment, the optical arrangement 116 includes an adaptive optic configured to adjust a working distance of the optical arrangement 116. In an embodiment, the optical arrangement 116 is located at least 50 mm, 100 mm, 150 mm, 200 mm, 250 mm, or 500 mm from the distal optical passage. The sensor 128 is further configured to generate a digital image of the dental tissue 120, as a function of the optical image of the dental tissue 120.

In an embodiment, the system 100 includes a laser, and a beam delivery system 140. The laser is configured to generate a laser beam as a function of a laser parameter. In some cases, the generation of the laser beam by the laser is performed as part of a laser treatment, for instance a preventative treatment such as without limitation cleaning, laser acid resistance, whitening, laser bacteria removal, and the like. In some embodiments, the laser parameter of the laser beam is controlled to affect treatment. Typically, the parameter of the laser beam is controlled to heat a portion of the surface of the dental hard tissue to a temperature within a range, for example between about 100° C. and about 1300° C. Exemplary laser parameters include pulse energy, pulse duration, peak power, average power, repetition rate, wavelength, duty cycle, laser focal region size, laser focal region location, and laser focal region scan speed. The laser beam performs a treatment on the dental tissue 120, as a function of the laser parameter. The beam delivery system 140 is in optical communication with the laser and the distal optical passage. The beam delivery system 140 is configured to deliver the laser beam from the laser to the hand piece 104. The hand piece 104 is configured to accept the laser beam from the beam delivery system 140 at the distal optical passage 108. The hand piece 104 is further configured to direct the laser beam, using the proximal optical passage, to the dental hard tissue. In an embodiment, the beam delivery system 140 includes at least a portion of the optical arrangement 116. In an embodiment, the sensor 128 has a shutter synchronized to pulses of the laser. In an embodiment, the beam delivery system 140 includes a beam scanning system configured to move the laser beam. According to an embodiment herein, the beam delivery system 140 may include a beam scanner. A beam scanner may include any optical component configured to actively scan the laser beam. Exemplary beam scanners include, without limitation, Risley prisms, spinning polygon mirrors, voice coil scanners (e.g., Part No. MR-15-30 from Optotune of Dietikon, Switzerland), galvanometers (e.g., Lightning II 2-axis scan head from Cambridge Technology of Bedford, Massachusetts, U.S.A.), and a gantry with a translating focus optic. Scanning methods related to dental laser systems are described in U.S. Pat. No. 9,408,673 by N. Monty et al., incorporated herein by reference. In some cases, beam scanner may be configured to scan the laser beam as a function of a scan parameter. Exemplary non-limiting scan parameters include scan patterns, scan jobs, jump speed, jump delay, mark speed, mark delay, dwell time, and the like. In some cases, scan parameters are a function of the laser parameters or vice versa. For example, in some cases a dwell time, duration of the beam scanner leaves the laser beam at a single point in a scan pattern, may be greater than the pulse duration of the laser beam, to limit beam smearing. Alternatively, or additionally, a scan pattern may be configured to have substantially no dwell time, and laser pulses are delivered while the beam scanner is in motion. The sensor 128 has a shutter synchronized to at least a movement of the beam scanning system. In an embodiment, the optical arrangement 116 includes an adaptive optic configured to adjust a working distance of the optical arrangement 116. The sensor 128 has shutter synchronized to the adaptive optic and the adjustment of the working distance of the optical arrangement 116. In additional system embodiments, the sensor 128 further includes a computing device 202 configured to control one or more of the laser, the sensor, the shutter, the adaptive optic, an illumination source 117 to generate illumination 119, and the beam scanning system.

The computing device 202 may be configured to receive dental parameter from the sensor and/or communicate the dental parameter to a remote device. A remote device may include a computing device located remotely from the system. Computing devices are described in greater detail below. In some cases, the computing device may be said to be in communication with the sensor and/or the remote device. Communication, for instance between a computing device and another device such as a sensor and/or remote device, may be performed by way of one or more networks and/or protocols. Exemplary communication networks and protocols are described in greater detail below. In some cases, communication may be performed using one or more signals; for instance, a signal may represent the dental parameter. Additional disclosure related to computing device and processing of digital dental images may be found in U.S. patent application Ser. No. 18/093,298 filed on Jan. 4, 2023 and incorporated by reference in this document in its entirety.

As used in this disclosure, a “signal” is any intelligible representation of data, for example from one device to another. A signal may include an optical signal, a hydraulic signal, a pneumatic signal, a mechanical, signal, an electric signal, a digital signal, an analog signal, and the like. In some cases, a signal may be used to communicate with a computing device, for example by way of one or more ports. In some cases, a signal may be transmitted and/or received by a computing device for example by way of an input/output port. An analog signal may be digitized, for example by way of an analog-to-digital converter. In some cases, an analog signal may be processed, for example by way of any analog signal processing steps described in this disclosure, before digitization. In some cases, a digital signal may be used to communicate between two or more devices, including without limitation computing devices. In some cases, a digital signal may be communicated by way of one or more communication protocols, including without limitation internet protocol (IP), controller area network (CAN) protocols, serial communication protocols (e.g., universal asynchronous receiver-transmitter [UART]), parallel communication protocols (e.g., IEEE [printer port]), and the like.

The system 100 may perform one or more signal processing steps on a signal. For instance, system 100 may analyze, modify, and/or synthesize a signal representative of data in order to improve the signal, for instance by improving transmission, storage efficiency, or signal to noise ratio. Exemplary methods of signal processing may include analog, continuous time, discrete, digital, nonlinear, and statistical. Analog signal processing may be performed on non-digitized or analog signals. Exemplary analog processes may include passive filters, active filters, additive mixers, integrators, delay lines, compandors, multipliers, voltage-controlled filters, voltage-controlled oscillators, and phase-locked loops. Continuous-time signal processing may be used, in some cases, to process signals which varying continuously within a domain, for instance time. Exemplary non-limiting continuous time processes may include time domain processing, frequency domain processing (Fourier transform), and complex frequency domain processing. Discrete time signal processing may be used when a signal is sampled non-continuously or at discrete time intervals (i.e., quantized in time). Analog discrete-time signal processing may process a signal using the following exemplary circuits sample and hold circuits, analog time-division multiplexers, analog delay lines and analog feedback shift registers. Digital signal processing may be used to process digitized discrete-time sampled signals. Commonly, digital signal processing may be performed by a computing device or other specialized digital circuits, such as without limitation an application specific integrated circuit (ASIC), a field-programmable gate array (FPGA), or a specialized digital signal processor (DSP). Digital signal processing may be used to perform any combination of typical arithmetical operations, including fixed-point and floating-point, real-valued and complex-valued, multiplication and addition. Digital signal processing may additionally operate circular buffers and lookup tables. Further non-limiting examples of algorithms that may be performed according to digital signal processing techniques include fast Fourier transform (FFT), finite impulse response (FIR) filter, infinite impulse response (IIR) filter, and adaptive filters such as the Wiener and Kalman filters. Statistical signal processing may be used to process a signal as a random function (i.e., a stochastic process), utilizing statistical properties. For instance, in some embodiments, a signal may be modeled with a probability distribution indicating noise, which then may be used to reduce noise in a processed signal.

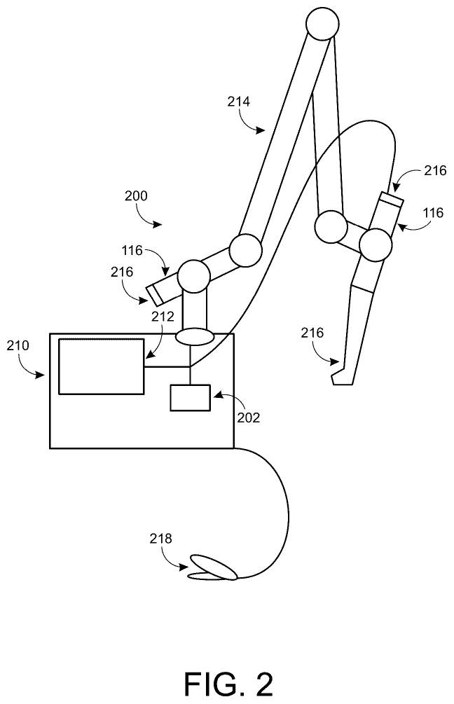

An exemplary system 200 is shown in FIG. 2. The exemplary system 200 includes a computing device 202, a console 210, and an image sensor 216. The console 210 houses components of the exemplary system 200, for example, a laser source to generate the laser beam, a direct current (DC) power supply to power the laser source, a beam shaper to shape an energy profile of the laser beam, a compressed air system to deliver compressed air for bulk cooling of dental hard tissue being treated, and a user interface 212 for user control. A beam delivery system 214 directs the laser beam to a hand piece 216. Exemplary beam delivery systems 214 include articulated arms, waveguides, and fiber optics. An exemplary articulated arm is provided by Laser Mechanisms of Novi, Michigan, U.S.A. The hand piece 216 is configured to be used intra-orally (i.e., within an oral cavity). In some cases, hand piece may 216 include a focus optic that converges laser beam to a focal region outside of the hand piece 216. In accordance with one embodiment, the system 200 is operated with a foot pedal 218, which is configured to initiate the laser source.

In accordance with one embodiment, the exemplary system 200 is used by a clinician. First, the clinician may input operating parameters into a user interface 212, for example by using a touch screen. Then the clinician places the hand piece 216 within a patient's mouth and directs the hand piece 216 toward dental hard tissue. For example, the clinician positions the hand piece 216 so that a focal region of the laser beam is coincident with or near (e.g., +/−1 mm, 2 mm, 3 mm, or 5 mm) a surface of a tooth or dental tissue. Then, the clinician activates the laser by stepping on a foot pedal 218. The clinician moves the hand piece 216 within the patient's mouth, carefully directing the focal region of the laser beam near every treatment surface of the patient's teeth.

To aid in practice of the claimed invention and parameter selection a table is provided below with exemplary ranges and nominal values for relevant parameters.

| Parameter | Min. | Max. | Nom. |
|---|---|---|---|
| Repetition Rate | 1 Hz | 10 KHz | 1 KHz |
| Pulse Energy | 1 μJ | 1 J | 10 mJ |
| Focal Region | 1 μm | 10 mm | 1 mm |
| Width | | | |
| Fluence | 0.01 J/cm² | 1 MJ/cm² | 1 J/cm² |
| Wavelength | 200-500 nm | 4000-12000 nm | 10.6 μm |
| Numerical | 0.00001 | 0.5 | 0.01 |
| Aperture (NA) | | | |
| Focal length | 10 mm | 1000 mm | 200 mm |
| Average Power | 1 mW | 100 W | 1 W |
| Peak Power | 50 mW | 5000 W | 500 W |
| Scan Speed | 0.001 mm/S | 100,000 mm/S | 10 mm/S |
| Scan Location | 0 | 10x Focal Region | 0.5x Focal Region |
| Spacing | | Width | Width |

| | |
|---|---|
| Polarization | Linear, circular, random, cylindrical, radial, |
| | tangential |
| Mode/Energy | Super-Gaussian, annular ring mode, TEM 01*, |
| Profile | flat-top, top-hat, Laguerre-Gaussian, Hermite- |
| | Gaussian, Bessel, Bessel-Gaussian |

Preventative dental treatment system 1000 delivers a radiation (e.g., laser beam) 1010 to a dental hard tissue 1012 (e.g., enamel, dentin, or cementum). The radiation is generated by a radiation source (e.g., laser source) 1014. An exemplary laser source 1014 is a carbon dioxide (CO₂) laser, for example, HPP DL-500 from Access Laser of Everett, Washington, U.S.A. Typically, the radiation source is selected to generate a radiation 1010 having a wavelength that is well absorbed (e.g., has an absorption coefficient greater than 1 cm⁻¹, 100 cm⁻¹, or 1,000 cm⁻¹) by the dental hard tissue 112. Exemplary wavelengths include wavelengths in either a first range between 200 and 500 nm and a second range between 4 and 12 μm. The radiation 1010 is delivered to a hand piece 1016 using a beam delivery system 1018. Exemplary beam delivery systems include articulating arms, fiber optics, and hollow wave guides. Certain exemplary articulating arms are provided by Laser Mechanisms of Novi, Michigan, U.S.A. The hand piece 1016 is configured to be used intra-orally (i.e., within an oral cavity). The hand piece comprises a distal optical passage. In many cases, distal optical passage may comprise an aperture; alternatively or additionally distal optical passage may include a coupling optic. The coupling optic 1020 accepts the radiation 1010 at a first face of the coupling optic. The radiation 1010 is transmitted within the coupling optic 1020. According to one embodiment, the radiation 1010 is ultimately ejected from a second face of the coupling optic. Exemplary coupling optics include windows, lenses, and prisms. Coupling optic in some embodiments comprises one of diamond, quartz (i.e., fused silica), glass, sapphire, zinc selenide, or zinc sulfide. In one embodiment the coupling optic 1020 is made out of diamond and produced by chemical vapor deposition (CVD).

Further description is provided below by way of certain exemplary embodiments. According to some embodiments, an ultraviolet (UV) laser source is used to produce a UV laser beam for treatment. Exemplary UV laser sources include diode-pumped solid-state (DPSS) lasers, frequency quadrupled Nd:YAG lasers, excimer lasers, and fiber lasers. An exemplary fiber laser series is the ULM/ULR-355 Series from IPG Photonics of Oxford, Massachusetts, U.S.A. The ULM/ULR-355 Series offers a 200 W average power system that operates in a quasi-continuous wave (CW) mode with a wavelength of 355 nm, a pulse duration of 1.4 nS, and repetition rate settings of 20, 40, and 80 MHz. Hydroxyapatite (HAP) has a relatively high absorption at 355 nm. The absorption coefficient of HAP at 355 nm is approximately 0.1 μm⁻¹ (i.e., 1000 cm⁻¹). To understand the potential for a UV laser source (e.g., ULM/ULR-355) a mathematical model is disclosed. The mathematical model assumes Beers absorption, Newtonian convection, and Fourier conduction. The model was coded using a nodal finite element analysis, which is described by J. Van de Ven et al. in “Laser Transmission Welding of Thermoplastics-Part I: Temperature and Pressure Modeling,” published in J. Manuf. Sci. Eng. In October of 2007 and incorporated herein by reference. The model was run assuming a 200 W irradiative power, 40% reflectivity between the air and enamel surface, 0.1 μm⁻¹ absorption coefficient, and a 1 mm 1/e² laser beam diameter at the enamel surface with a Gaussian profile.

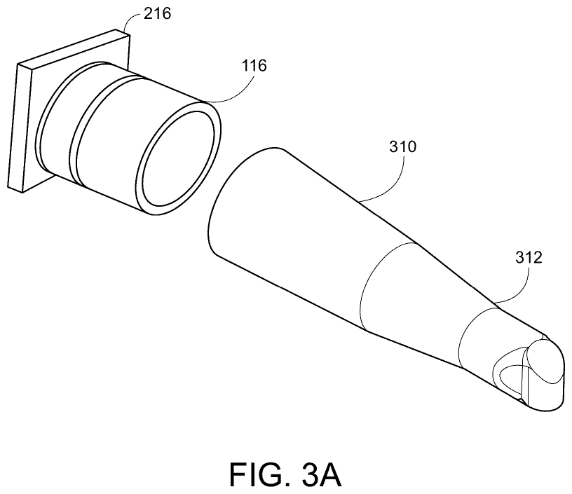

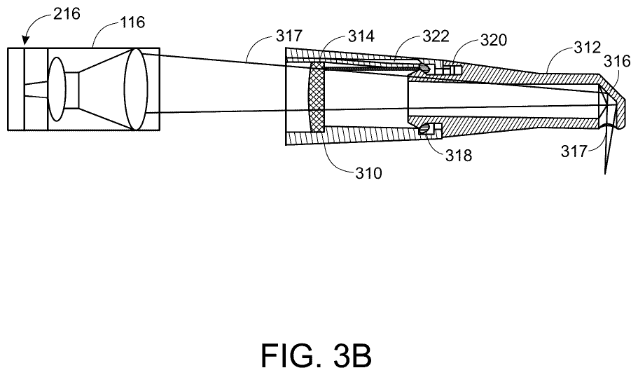

FIG. 3A illustrates a view of a dental laser hand piece 310 with an attachable (i.e., removable) tip 312, in accordance with an embodiment of the present subject matter. FIG. 3B illustrates a cross-sectional view of a dental laser hand piece 310 with an attachable tip 312 of FIG. 3A, in accordance with an embodiment of the present subject matter. Within the hand piece 310, a focus optic 314 may be located to converge a laser beam. Opposite the focus optic 314, a reflector 316 may be positioned within the attachment 312. The reflector 316 may have a high reflectivity (e.g., at least as great as 50%) at a wavelength of a laser beam or sensed electromagnetic radiation. The reflector 316 may be positioned to reflect a laser beam out of an aperture (i.e., distal optical passage) 317 within attachment 312. In some cases, the attachment is made from an ejection-molded polymer. The reflector 316, in some embodiments, is coated on a surface of the attachment 312. Alternatively, the reflector 316 comprises a separate substrate from the attachment 316. Exemplary coatings for the reflector 316 include broadband coatings (e.g., silver, protected silver, and gold) and dielectric coatings. The attachment 312, in certain exemplary embodiments shown in FIGS. 3A-B, is attached to the hand piece 310 using a canted coil spring 318 (e.g., Bal-Seal of Foothill Ranch, California U.S.A.). The canted coil spring 318, in some cases, can also provide an electrical connection between a one-wire authentication chip 320 storing the machine-readable code and an electrical connection in the hand piece 322, which is ultimately connected to a processor within the laser treatment system. An exemplary one-wire authentication chip is Part No. DS28C50 from Maxim Integrated of San Jose, California, U.S.A. The attachment 312 is shown in FIGS. 3A-B helps direct the laser beam intra-orally by reflecting the laser beam. In other embodiments, a consumable attachment is provided that does not reflect the laser beam.

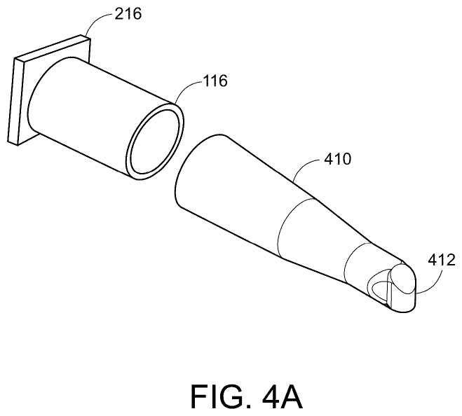

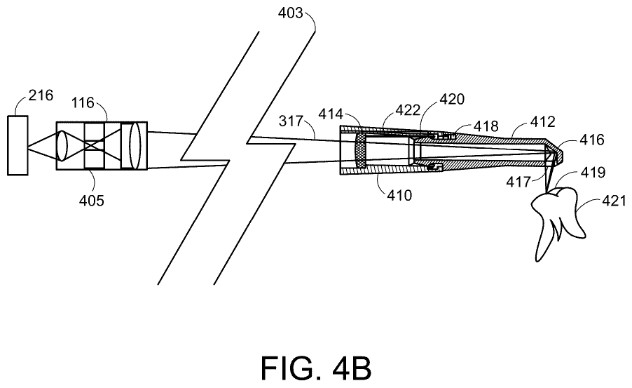

FIG. 4A illustrates a hand piece 410 with a consumable sheath 412 attached, in accordance with an embodiment of the present subject matter. The consumable sheath 412 allows for intra-oral use without the need for sterilization of the hand piece 410 in between patients. This is because only the sheath 412 (during normal use) comes into contact with a patient. A new sheath therefore is used with each patient (and, therefore each new treatment). FIG. 4B illustrates a cross-sectional view of the hand piece 410 and the consumable sheath 412 of FIG. 4A. A focus optic 414 may be positioned within the hand piece 410. The focus optic 414 may be configured to converge a laser beam. Opposite and down-beam from the focus optic 414 may be a reflector 416. Unlike the embodiment illustrated in FIGS. 4A-B, the reflector 416 is not a part of the consumable attachment (the sheath) 412. Instead, the reflector 416 is an integrated component of the hand piece 410. The reflector 416 in some cases is a separate part from the rest of the hand piece 410 and is therefore removable for maintenance. However, the reflector 416 in this case is not intended for removal with each treatment. The reflector 416 is configured to reflect the converging laser beam out of an aperture 417 within the sheath 412. The reflector 416 is pointed to an object 419 present on the. tooth 421. In some embodiments, the sheath 412 is attached to the hand piece in FIG. 4B by a snap feature (not shown). Alternative attachment of the sheath and the consumable attachment are also envisioned, for example, fasteners, threads, clamps, magnets, and O-rings. The sheath 416, in some embodiments, comprises a one-wire authentication chip 418. The chip may include machine-readable code. FIG. 4B further depicts a break 403 indicative of a distance or orientation between the optical arrangement 116, and imaging channel 317. The optical arrangement 116 includes various adaptive optics 405. The hand piece 410 comprises a pogo pin 420 which is configured to make an electrical contact between the chip 418 and an electrical connection 422 within the hand piece 410. The electrical connection within the hand piece communicates with a processor in the laser treatment system, which verifies the machine-readable code. In another exemplary embodiment, the sheath comprises the machine-readable code in a different medium, for example, a barcode, a 2D barcode, or an RFID tag.

To aid in practice of the claimed invention and parameter selection a table is provided below with exemplary ranges and nominal values for relevant parameters.

| Parameter | Min. | Max. | Nom. |
|---|---|---|---|
| Repetition Rate | 1 Hz | 10 KHz | 1 KHz |
| Pulse Energy | 1 μJ | 1 J | 10 mJ |
| Focal Region Width | 1 μm | 10 mm | 1 mm |
| Fluence | 0.01 J/cm² | 1 MJ/cm² | 1 J/cm² |
| Wavelength | 200-500 nm | 4000-12000 nm | 10.6 μm |
| Numerical Aperture | 0.00001 | 0.5 | 0.01 |
| (NA) | | | |
| Focal length | 10 mm | 1000 mm | 200 mm |
| Average Power | 1 mW | 100 W | 1 W |
| Peak Power | 50 mW | 5000 W | 500 W |
| Scan Speed | 0.001 mm/S | 10 mm/S | 100,000 mm/S |
| Scan Location | 0 | 0.5 x Focal Region | 10x Focal Region |
| Spacing | | Width | Width |

| | |
|---|---|
| Machine Readable | Barcode, 2D Barcode, RFID Tag, Authentication Chip (e.g., One-Wire), |
| Code Mediums | Digital, Software Token (e.g., Google Authenticator), Paper Token (e.g., |
| | Transaction Authorization Number [TAN]), a consumable intra-oral |
| | component (e.g., hand piece attachment), and a film. |
| Machine Readable | One Time Password (OTP), Hash Message Authentication Code, Digest, |
| Code Contents | Transaction Authorization Number (TAN), are Encoded; are Encrypted; |
| | are Obfuscated (i.e., identification of valid coupons is held in secret); and, |
| | a digital signature. |
| Fluoride Treatment | Sodium Fluoride, Stannous Fluoride, Titanium Tetrafluoride, |
| Ingredients | Acidulated-Phosphate Fluoride, and Amine Fluoride |

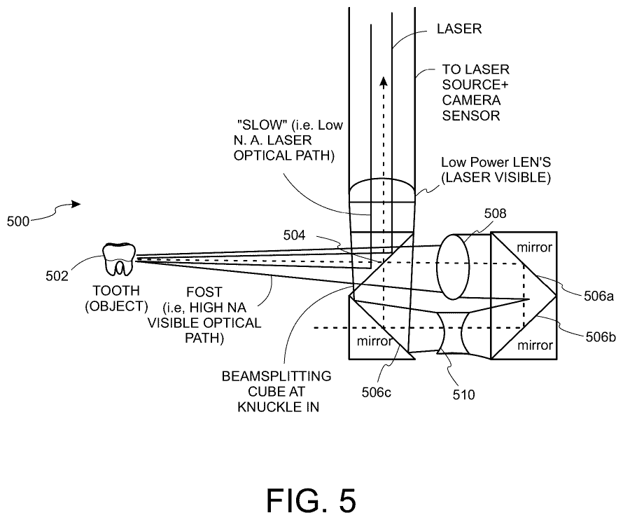

FIG. 5 illustrates a exemplary system 500 for tooth imaging, in accordance with an embodiment of the present subject matter. FIG. 5 depicts an object i.e. tooth 502, a beam splitter 504, reflectors 506a-c, and beam shaping optics 508 and 510. In some cases, beam splitter may include a dichroic optic configured to selectively pass and/or reflect as a function of wavelength. For instance, in some cases beam splitter may be configured to reflect laser wavelength light (e.g., 9-12 micron) and transmit imaging channel wavelength light (e.g., visible spectrum). In some cases, beam splitter may include a cube comprising two right prisms with a beam splitting surface (i.e., selectively reflective) along a hypotenuse face. In some cases, having a cube rather than a plate beam splitter surface may limit off axis aberrations, such as deflection, decenter, astigmatism, and coma.

With continued reference to FIG. 5, imaging channel (originating from object) may pass through beam splitter 504 a number of lenses 508 and 510, be reflected by a number of reflectors 506a-c, and transmit back through the beamsplitter 504 re-combining with laser beam. In some cases, a lens may be placed up beam from beamsplitter 504 (nearer to laser source); in this case, the lens will act on both the imaging channel and the laser beam. In some embodiments, beam splitter 504 may allow imaging channel to have a higher numerical aperture than laser. For instance. Optics 508 and 510 may be placed in imaging channel outside of laser beam path. Decoupling imaging channel from laser channel allows for an increased numerical aperture for the imaging channel. Increased numerical aperture improves resolution of the imaging channel. Laser beam however, in some case, must maintain a relatively low numerical aperture in order to ensure energy dosage within a therapeutic range. See for example, co-owned and co-invented U.S. Pat. No. 11,607,297 titled “PREVENTATIVE DENTAL HARD TISSUE LASER TREATMENT SYSTEMS AND METHODS,” granted Mar. 21, 2023 and incorporated by reference in this document in its entirety.

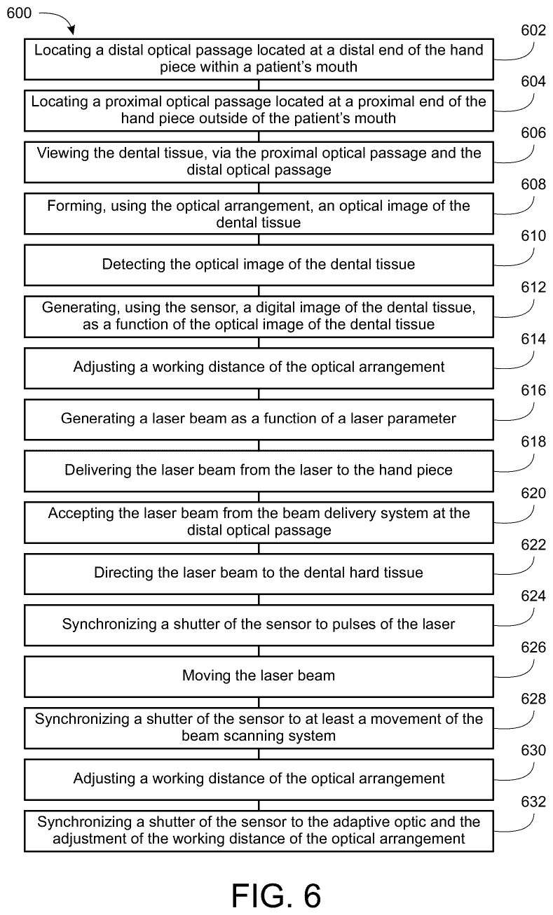

FIG. 6 illustrates a flow diagram illustrating a method 600 for oral imaging, in accordance with an embodiment of the present subject matter. The method 600 includes a step 602 of locating, using a hand piece, a distal optical passage located at a distal end of the hand piece within a patient's mouth. The method 600 includes a step 604 of locating, using the hand piece, a proximal optical passage located at a proximal end of the hand piece outside of the patient's mouth. The method 600 includes a step 606 of viewing at the dental tissue using an optical arrangement connected to both the proximal and distal optical passages. In additional method embodiments, the optical arrangement includes a telecentric lens. In additional method embodiments, the optical arrangement is located at least 50 mm, 100 mm, 150 mm, 200 mm, 250 mm, or 500 mm from the distal optical passage. The method 600 includes a step 608 of forming, using the optical arrangement, an optical image of the dental tissue. The method 600 includes a step 610 of detecting, using a sensor in optical communication with the optical arrangement, the optical image of the dental tissue. The method 600 includes a step 612 of generating, using the sensor, a digital image of the dental tissue, as a function of the optical image of the dental tissue. In additional method embodiments, the method 600 includes a step 614 of adjusting, using the optical arrangement comprising an adaptive optic, a working distance of the optical arrangement. The method 600 further includes a step 616 of generating, using a laser, a laser beam as a function of a laser parameter. The laser beam performs a treatment on the dental tissue, as a function of the laser parameter. Further, the method 600 includes a step 618 of delivering the laser beam from the laser to the hand piece by using a beam delivery system. In additional method embodiments, the method 600 includes a step 620 of accepting, using the hand piece, the laser beam from the beam delivery system at the distal optical passage. In additional method embodiments, the beam delivery system includes at least a portion of the optical arrangement. In additional method embodiments, the method 600 includes a step 622 of directing the laser beam, using the proximal optical passage of the hand piece, to the dental hard tissue. In additional method embodiments, the method 600 includes a step 624 of synchronizing a shutter of the sensor to pulses of the laser. In additional method embodiments, the method 600 includes a step 626 of moving, using a beam scanning system of the beam delivery system, the laser beam. In additional method embodiments, the method 600 includes a step 628 of synchronizing a shutter of the sensor to at least a movement of the beam scanning system. In additional method embodiments, the method 600 includes a step 630 of adjusting a working distance of the optical arrangement, using an adaptive optic. In additional method embodiments, the method 600 includes a step 632 of synchronizing a shutter of the sensor to the adaptive optic and the adjustment of the working distance of the optical arrangement. In additional method embodiments, the sensor further includes a computing device configured to control one or more of the laser, the sensor, the shutter, the adaptive optic, an illumination source 117, and the beam scanning system.

### Exemplary Beam Delivery and Imaging Project

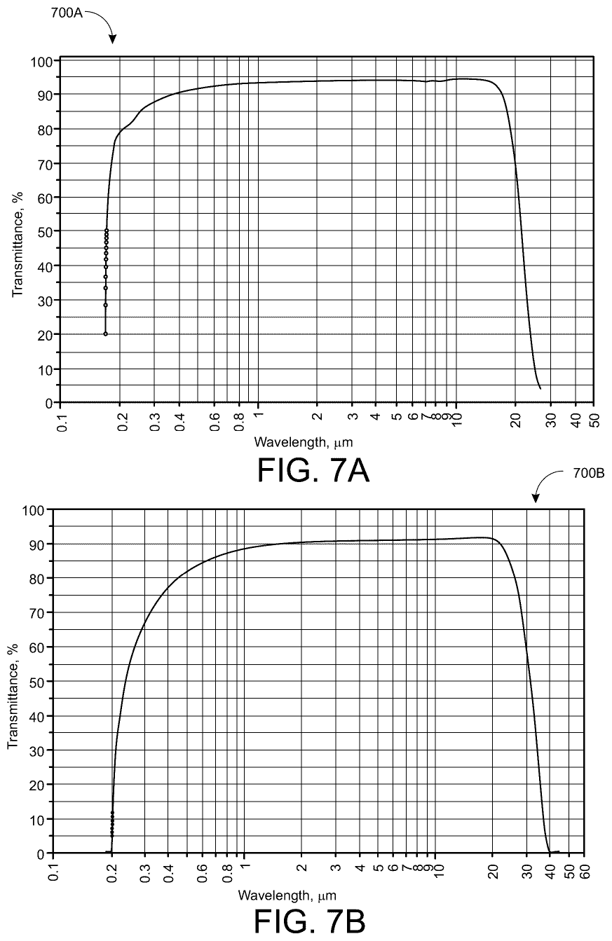

FIGS. 7-18 show an exemplary design project for delivery an infrared laser beam (e.g., 9-12 micron) to an object and imaging the object in another wavelength (e.g., visible spectrum). Initially, only few optical materials may transmit both infrared and visible wavelengths. Given the objective of transmitting both visible and IR light through the system, the options for glass selection are considerably constrained. After evaluating transmittance within both the 400-700 nm and 10.6 μm ranges, the choices were narrowed down to three types of glass: CLEARTRAN (i.e., ZnS), and salts optical materials, such as KCl, and KBr. The latter two varieties exhibit brittleness and slight solubility in water, necessitating careful handling. FIGS. 7A-7B illustrate graphical representations 700A and 700B of transmission data of KCl and KBr, in accordance with an embodiment of the present subject matter.

The current project includes a design of an 8f CO₂ beam steering optics. In some embodiments, beam scanning system may be located before an input to the beam delivery system, e.g., articulating arm. In this case, the tip/tilt gradients introduced by the beam scanning system may be relayed through the beam delivery system by relay lenses, e.g. 8F relay lens arrangement. In some cases, lenses for these beam steering optics may be biconvex This choice was based on the requirement to simultaneously image the aperture and the beam. Consequently, the lens must maintain an invariant orientation to ensure consistent imaging over the same distance, resulting in identical working distances at the front and back.

A request was made to modify the lens shapes to plano-convex; however, due to the far IR wavelengths, the biconvex lenses still performed satisfactorily. In some cases, imaging through relay optics may use a symmetrical lens design is necessary. In some cases, each lens may be achromatized, requiring the use of at least two types of glass per lens. In some cases, if a symmetrical layout is required, this may necessitates an increase to three glass types per lens. Alternatively, lenses may not be symmetric or achromatized.

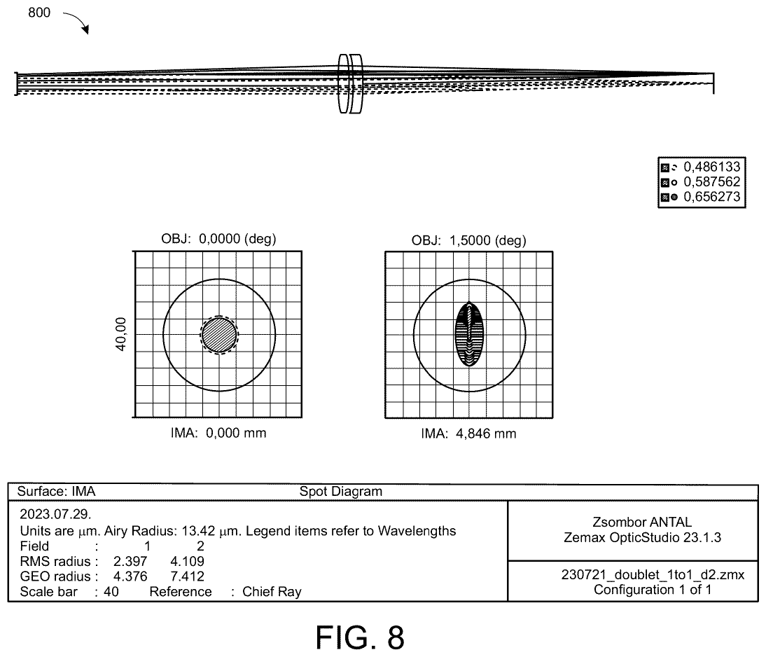

Throughout the design process, the physical size of the beam steering system posed a significant constraint. The current model features a 9.9 mm aperture and operates with a 1.5° off-axis field. Performance remains diffraction-limited across all wavelengths, yielding minimal wavefront error, even at 10.6 μm. FIG. 8 illustrates a layout and spot diagram 800 of the separated KBr-CLEARTRAN achromat, in accordance with an embodiment of the present subject matter.

The optical scheme described above was duplicated and reversed to create a 4f relay system. Further optimizations were performed to enhance image quality and facilitate manufacturing. The first lens (KBr) is biconvex, with R1=−R2, both possessing positive power. The second lens (CLEARTRAN) shares the same radii as R1 on the front but exhibits negative power. Both lenses have a thickness of 5 mm, and their spacing remains constant along the facing sides at 2 mm, dictated by the aforementioned radii condition.

Their performance remains robust, with the RMS radius approximately 25% of the Airy radius in angular space. RMS wavefront was measured in the conjugate plane of the pupil for each wavelength. The system maintains the diffraction limit. However, due to incomplete telecentricity, the conjugate of the pupil shifts approximately 30 mm forward.

| Wavelength | RMS wavefront |
|---|---|
| [um] | [wavelengths] |
| 0.486 | 0.0581 |
| 0.587 | 0.014 |
| 0.656 | 0.0397 |
| 10.6 | 0.0162 |

In some embodiments, beam steering relay lenses and imaging channel may be combined in order to allow for placement of camera and sensor away from handpiece and patient's mouth. A (dichroic) mirror, positioned 20 mm in front of the conjugate plane, ensures that the reflected beam's conjugate pupil is 20 mm beyond the mirror's surface, which is then designated as the aperture stop.

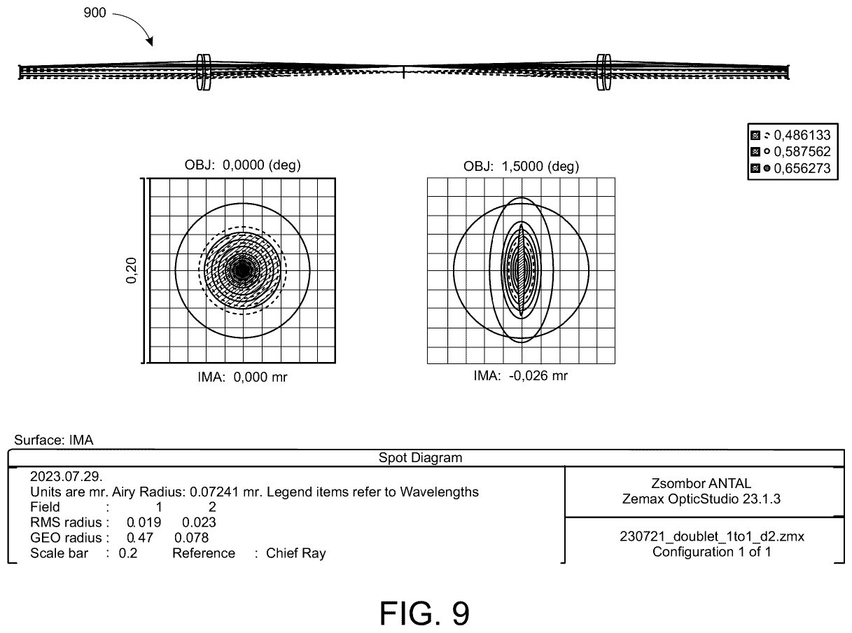

FIG. 9 illustrates a layout and spot diagram 900 of the separated KBr-CLEARTRAN achromat on angular space, in accordance with an embodiment of the present subject matter. The focusing lenses on the image side consist of two off-the-shelf achromats from Thorlabs, specifically AC254-400-A, resulting in an effective focal length (EFL) of 200 mm. On the object side, a custom-made 200 mm achromat composed of KCl and CLEARTRAN is utilized. The ratio of the focal lengths is 1, indicating that the optical system functions as an image relay. The field of view (FoV) is 10×10 mm. Off-the-shelf lenses were selected to enhance the transmission of visible light.

The modulation transfer function (MTF) drops to 20% at 33 lp/mm, with 66 lines present within 1 mm, making 15 μm structures still discernible. Zonal fields exhibit improved MTF values by 15-20% due to field curvature and spherical aberration. Field curvature necessitates a compromise between on-axis and off-axis field points.

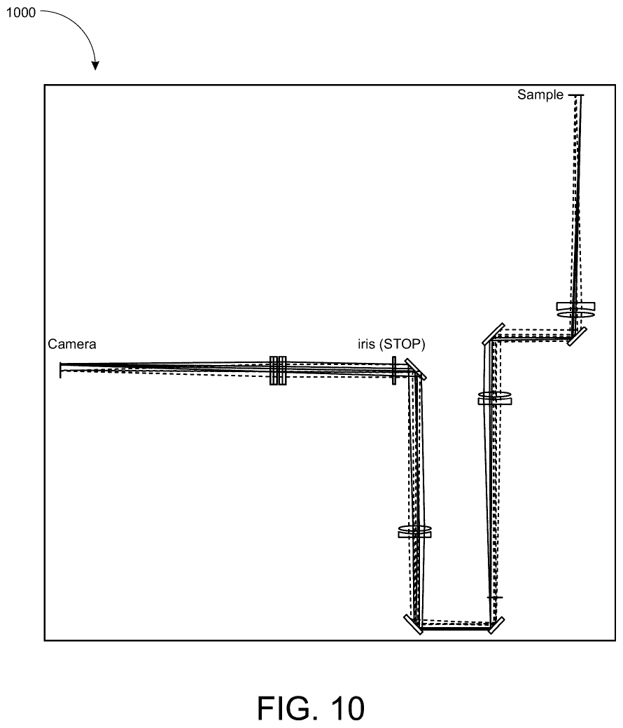

FIG. 10 illustrates a layout 1000 of an original system with the redesigned lens, in accordance with an embodiment of the present subject matter. The CO₂ laser spot size remains within the diffraction limit at all galvo angles. The distance from the last lens to the sample is 192.7 mm.

| Galvo tilt [°] | | Image plane position | |
|---|---|---|---|
| 1st (x) | 2nd (y) | x-pos [mm] | y-pos [mm] |
| 0 | 0 | 0 | 0 |
| 0 | 0.5 | 0 | 3.44 |
| 0 | 1 | 0 | 6.902 |
| 0.5 | 0 | 3.464 | 0 |
| 0.5 | 0.5 | 3.466 | 3.446 |
| 0.5 | 1 | 3.471 | 6.904 |
| 1 | 0 | 6.934 | 0 |
| 1 | 0.5 | 6.938 | 3.449 |
| 1 | 1 | 6.948 | 6.91 |

The above table depicts CO₂ laser positions as a function of galvo tilt.

On the image plane, grid points averaged a shrinkage of 1.6% along the x-axis and 2.2% along the y-axis. Consequently, the object area scanned by the laser is approximately 200 μm shorter along the x-axis and 300 μm shorter along the y-axis. This roughly corresponds to the Airy radius of the beam in its focal plane.

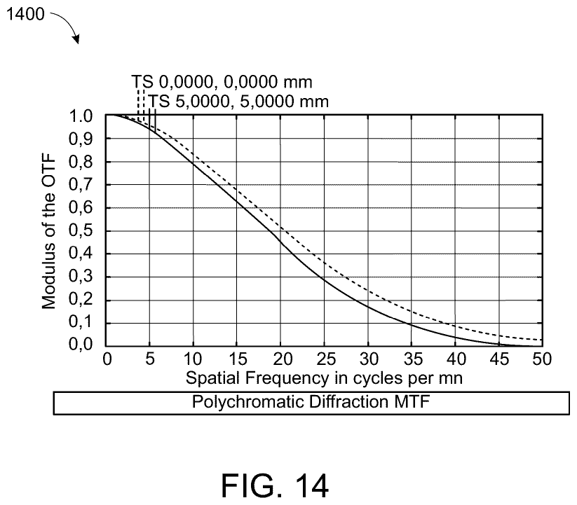

In this scenario, all four relay lenses are custom-designed achromats, which significantly impacts transmission since CLEARTRAN typically averages around 70%. However, this could be rectified with AR coating (or VIS coating if sufficient laser power is available). The tube lenses remain the same Thorlabs achromatic lenses as in the previous case, although performance slightly degraded, likely due to passing through another 4f relay system. MTF values average 20% at 25 lp/mm, as shown in FIG. 14. Considering the complexity of positioning an imaging channel 317 (shown in FIG. 3B) at this juncture, its performance is surprisingly good.

A number of different camera sensor locations were considered as a part of this project. O1, O2, O3, and O4 all represent different potential locations for the image plan of the imaging channel. These positions and their related optical performances are discussed below.

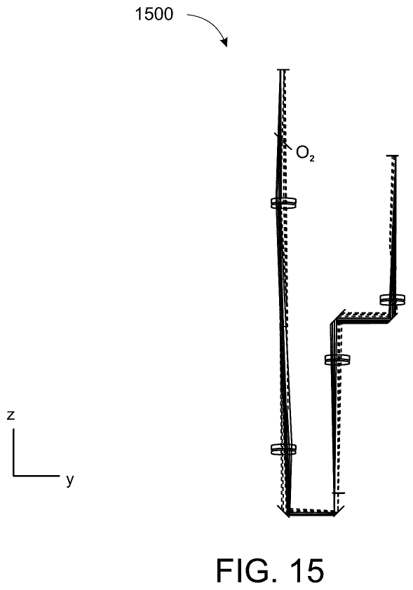

A minor adjustment is made concerning O2, as its original position was suboptimal, resulting in beam divergence at the initial O2 location. Consequently, a tube lens with stronger power would have been required in front of the camera. O2 is modified to allow light to pass through the 3rd relay lens. By substituting the CO₂ steering mirror with a dichroic one, VIS light can be transmitted through M2, with the 3rd relay lens handling the focusing, as depicted in FIG. 15.

The focal length of the relay lens is now 15 mm shorter than before, resulting in a slight demagnification of about 8%. MTF averages 20% at 27 lp/mm, although differences in contrast between the center and corners of the image are not negligible. The center exhibits almost twice the quality above 15 lp/mm. This discrepancy can be minimized by adjusting the working distance on either the object or image side. CO₂ beam steering functions adequately, with the galvo scan grid values changing by less than 2.7%.

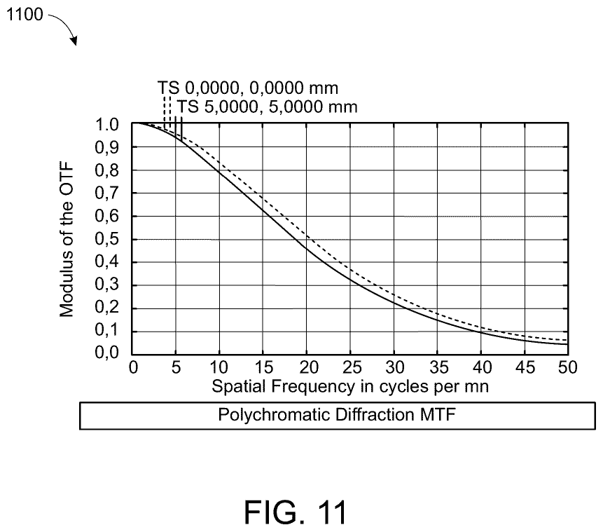

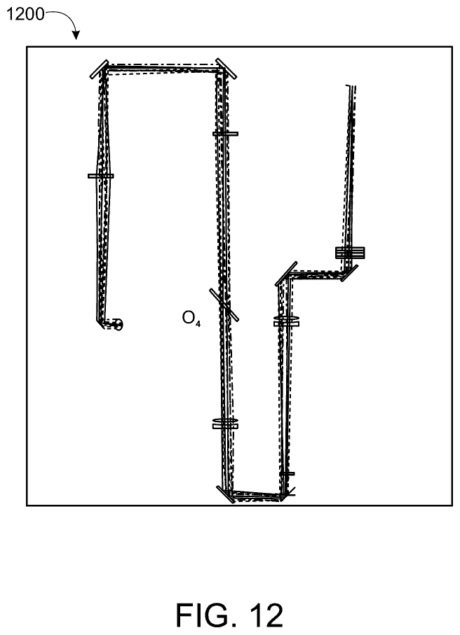

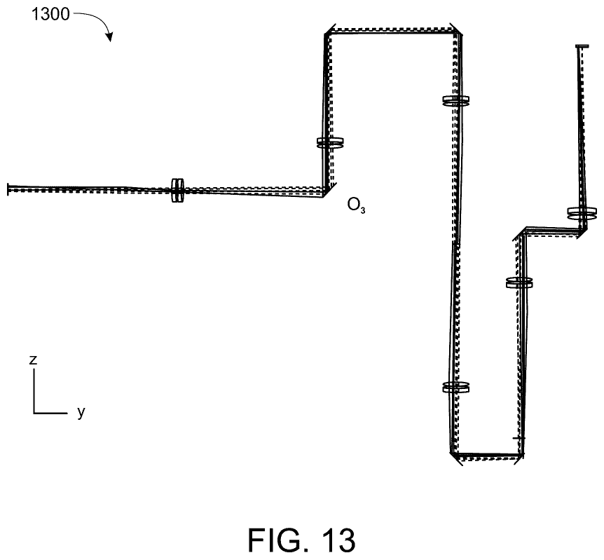

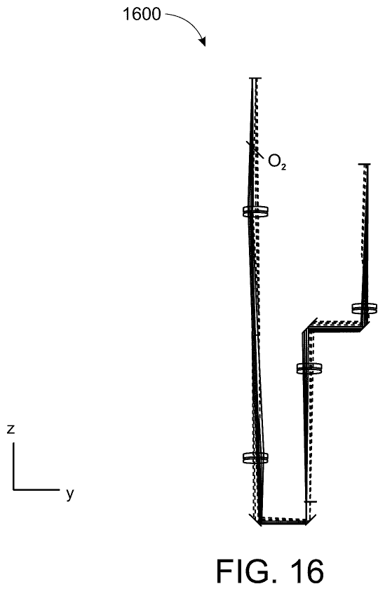

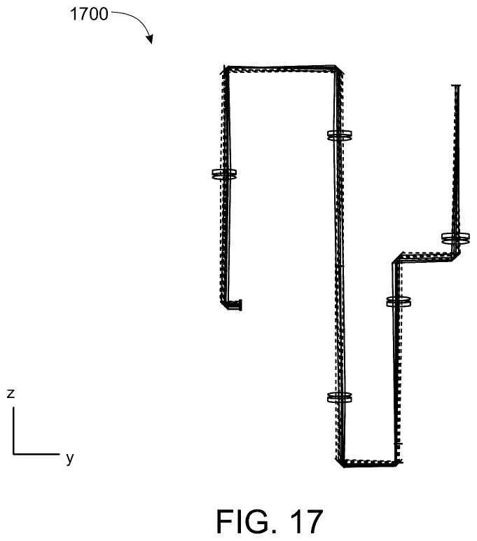

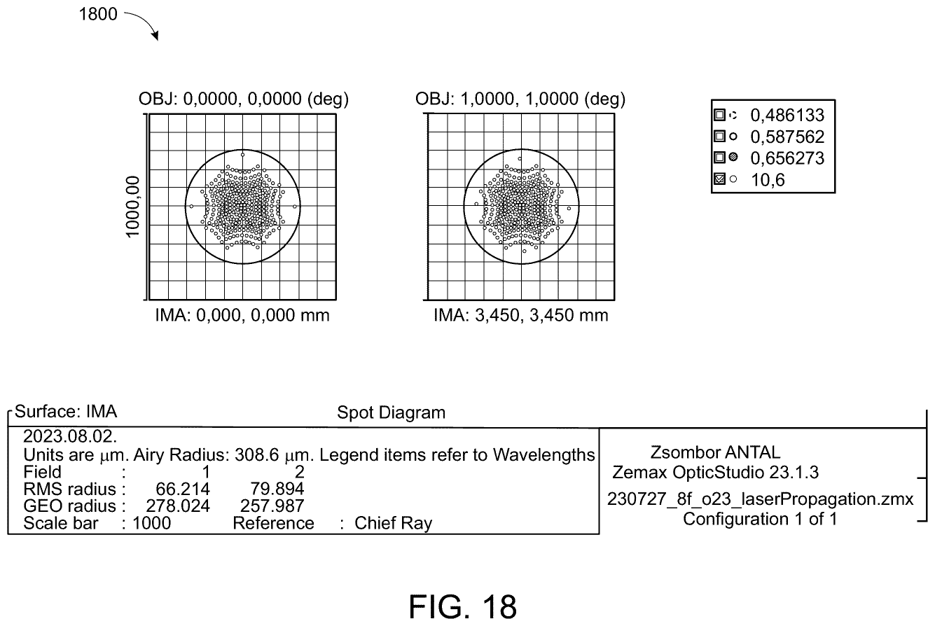

FIG. 11 illustrates a graphical representation 1100 of poly MTF of the original system with the achromatized lens, in accordance with an embodiment of the present subject matter. FIG. 12 illustrates a layout 1200 of the combined 8f relay system, in accordance with an embodiment of the present subject matter. FIG. 13 illustrates a layout 1300 of case O3, where the mirror for imaging purpose is placed after the 4th relay lens, in accordance with an embodiment of the present subject matter. FIG. 14 illustrates a graphical representation 1400 of poly MTF of case O3, in accordance with an embodiment of the present subject matter. FIG. 15 illustrates a layout 1500 of case O2, where the relay lens is responsible for imaging, in accordance with an embodiment of the present subject matter. FIG. 16 illustrates a perspective view 1600 of a poly MTF of case O2, in accordance with an embodiment of the present subject matter. FIG. 17 illustrates a layout 1700 of the combined 8f relay system with all relay lens replaced by doublets, in accordance with an embodiment of the present subject matter. FIG. 18 illustrates a spot diagram 1800 of the CO₂ beam through the achromatic 8f system, in accordance with an embodiment of the present subject matter.

Computing device may include any computing device as described in this disclosure, including without limitation a microcontroller, microprocessor, digital signal processor (DSP), and/or system on a chip (SoC) as described in this disclosure. The computing device may include, be included in, and/or communicate with a mobile device such as a mobile telephone or smartphone. Computing device may include a single computing device operating independently, or may include two or more computing devices operating in concert, in parallel, sequentially, or the like; two or more computing devices may be included together in a single computing device or two or more computing devices. Computing device may interface or communicate with one or more additional devices as described below in further detail via a network interface device. The network interface device may be utilized for connecting computing device to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus, or other relatively small geographic space), a telephone network, a data network associated with a telephone/voice provider (e.g., a mobile communications provider data and/or voice network), a direct connection between two computing devices, and any combinations thereof. A network may employ a wired and/or wireless mode of communication. In general, any network topology may be used. Information (e.g., data, software, etc.) may be communicated to and/or from a computer and/or a computing device. Computing device may include but is not limited to, for example, a computing device or cluster of computing devices in a first location and a second computing device or cluster of computing devices in a second location. Computing device may include one or more computing devices dedicated to data storage, security, distribution of traffic for load balancing, and the like. Computing device may distribute one or more computing tasks as described below across a plurality of computing devices of computing device, which may operate in parallel, in series, redundantly, or in any other manner used for the distribution of tasks or memory between computing devices. Computing device may be implemented using a “shared nothing” architecture in which data is cached at the worker, in an embodiment, this may enable the scalability of system 100 and/or the computing device.

Further, computing device may be designed and/or configured to perform any method, method step, or sequence of method steps in any embodiment described in this disclosure, in any order and with any degree of repetition. For instance, computing device may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device may perform any step or sequence of steps as described in this disclosure in parallels, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within the ordinary skill to achieve methods, and systems, according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions, and additions may be made to that which is specifically disclosed herein without departing from the spirit and scope of the present invention.

What is claimed is:

1. A system for oral imaging, the system comprising:
a hand piece comprising:
a distal optical passage located at a distal end of the hand piece configured to be located within a patient's mouth; and
a proximal optical passage located at a proximal end of the hand piece configured to be located outside of the patient's mouth;
an optical arrangement in optical communication with the proximal optical passage and the distal optical passage, wherein the optical arrangement is configured to:
view dental tissue, via the proximal optical passage and the distal optical passage; and
form an optical image of the dental tissue; and
a sensor in optical communication with the optical arrangement and configured to:
detect the optical image of the dental tissue; and
generate a digital image of the dental tissue, as a function of the optical image of the dental tissue.

2. The system of claim 1, wherein the optical arrangement comprises a telecentric optical.

3. The system of claim 1, wherein the optical arrangement comprises an adaptive optic configured to adjust a working distance of the optical arrangement.

4. The system of claim 1, wherein the optical arrangement is located at least 50 mm, 100 mm, 150 mm, 200 mm, 250 mm, or 500 mm from the distal optical passage.

5. The system of claim 1, further comprising:
a laser configured to generate a laser beam as a function of a laser parameter, wherein the laser beam performs a treatment on the dental tissue, as a function of the laser parameter; and
a beam delivery system in optical communication with the laser and the distal optical passage, wherein the beam delivery system is configured to deliver the laser beam from the laser to the hand piece; and
wherein the hand piece is further configured to:
accept the laser beam from the beam delivery system at the distal optical passage; and
direct the laser beam, using the proximal optical passage, to the dental hard tissue.

6. The system of claim 5, wherein the beam delivery system comprises at least a portion of the optical arrangement.

7. The system of claim 5, wherein the sensor has a shutter synchronized to pulses of the laser.

8. The system of claim 5, wherein the beam delivery system comprises a beam scanning system configured to move the laser beam; and
wherein the sensor has a shutter synchronized to at least a movement of the beam scanning system.

9. The system of claim 8, wherein:
the optical arrangement comprises an adaptive optic configured to adjust a working distance of the optical arrangement; and
the sensor has shutter synchronized to the adaptive optic and the adjustment of the working distance of the optical arrangement.

10. The system of claim 8, further comprising at least a computing device configured to control one or more of the laser, the sensor, the shutter, the adaptive optic, an illumination source, and the beam scanning system.

11. A method for oral imaging, the method comprising:
locating, using a hand piece, a distal optical passage located at a distal end of the hand piece within a patient's mouth;
locating, using the hand piece, a proximal optical passage located at a proximal end of the hand piece outside of the patient's mouth;
viewing, using an optical arrangement in optical communication with the proximal optical passage and the distal optical passage, the dental tissue, via the proximal optical passage and the distal optical passage;
forming, using the optical arrangement, an optical image of the dental tissue;
detecting, using a sensor in optical communication with the optical arrangement, the optical image of the dental tissue; and
generating, using the sensor, a digital image of the dental tissue, as a function of the optical image of the dental tissue.

12. The method of claim 11, wherein the optical arrangement comprises a telecentric optical.

13. The method of claim 11, further comprising adjusting, using the optical arrangement comprising an adaptive optic, a working distance of the optical arrangement.

14. The method of claim 11, wherein the optical arrangement is located at least 50 mm, 100 mm, 150 mm, 200 mm, 250 mm, or 500 mm from the distal optical passage.

15. The method of claim 11, further comprising:
generating, using a laser, a laser beam as a function of a laser parameter, wherein the laser beam performs a treatment on the dental tissue, as a function of the laser parameter;
delivering, using a beam delivery system in optical communication with the laser and the distal optical passage, the laser beam from the laser to the hand piece;
accepting, using the hand piece, the laser beam from the beam delivery system at the distal optical passage; and
directing the laser beam, using the proximal optical passage of the hand piece, to the dental hard tissue.

16. The method of claim 15, wherein the beam delivery system comprises at least a portion of the optical arrangement.

17. The method of claim 15, further comprising synchronizing a shutter of the sensor to pulses of the laser.

18. The method of claim 15, further comprising:
moving, using a beam scanning system of the beam delivery system, the laser beam; and
synchronizing a shutter of the sensor to at least a movement of the beam scanning system.

19. The method of claim 15, further comprising:
adjusting a working distance of the optical arrangement, using an adaptive optic; and
synchronizing a shutter of the sensor to the adaptive optic and the adjustment of the working distance of the optical arrangement.

20. The method of claim 19, further comprising controlling, using at least a computing device, one or more of the laser, the sensor, the shutter, the adaptive optic, an illumination source, and the beam scanning system.
